(19) United States
(12) Patent Application Publication
(10) Pub. No.: US 2006/0112698 A1
(43) Pub. Date: Jun. 1, 2006

(54) Medical configuration of vortex tubes and method of use

(75) Inventors: Karl Hans Cazzini, Orchard Park, NY (US); Joel T. Jusiak, Holland, NY (US)

Correspondence Address: Kevin D. McCarthy;Roach Brown McCarthy & Gruber, P.C., 1620 Liberty Building, 420 Main Street, Buffalo, NY, 14202, US

(73) Assignee: Gaymar Industries, Inc.

(21) Appl. No.: 11/000,836

(22) Filed: Dec. 1, 2004

(51) Int. Cl. F25B 9/02; A61F 7/00; A61F 7/12; F25D 9/00

(52) U.S. Cl. 62/5; 607/96

(57) ABSTRACT

The present invention is directed to a vortex tube assembly. The vortex tube assembly has a compressed fluid gas inlet. When the compressed fluid circulates in the vortex tube, the compressed fluid forms a cold end on the vortex tube that expels cold thermal energy and a warm end on the vortex tube that expels warm thermal energy. The present invention also has an applicator attached to a vortex tube to distribute thermal energy from the vortex tube to a patient to increase or decrease at least a portion of the patient's temperature without damaging the patient's skin.

### FIELD OF THE INVENTION

The present invention is directed to the use of a vortex tube.

### BACKGROUND OF THE INVENTION

a. Vortex Tubes

The vortex tube was invented in 1928 by George Ranque. The vortex tube uses compressed air as a power source, has no moving parts, and produces hot air from one end and cold air from the other. The volume and temperature of these two airstreams are adjustable with a valve built into the hot air exhaust. Temperatures as low as −50° F. (−46° C.) and as high as +260° F. (127° C.) are possible.

Theories abound regarding the dynamics of a vortex tube. One widely accepted explanation of the phenomenon is that, as shown in FIG. 1, compressed air 2 (solid line) is supplied to the vortex tube 10 and passes through nozzles 3 that are tangent to an internal counterbore. These nozzles set the air in a vortex motion 4 (dashed lines). This spinning stream of air turns 90° and passes down the hot tube 5 in the form of a spinning shell, similar to a tornado. A valve 6 at one end of the tube allows some of the warmed air 7 (dashed arrow lines) to escape through the warm end 14 of the vortex tube 10. What does not escape heads back down the tube 5 as a second vortex 8 (long dashed-dot line) inside the low pressure area of the larger vortex. This inner vortex 8 loses heat and exhausts through the other end (a.k.a. cold end 16 of the vortex tube 10) as cold air.

While one airstream moves up the tube and the other airstream down the tube, both rotate in the same direction at the same angular velocity. That is, a particle in the inner stream completes one rotation in the same amount of time as a particle in the outer stream. Due to the principle of conservation of angular momentum, it is expected that the rotational speed of the smaller vortex might increase. But in the vortex tube, the speed of the inner vortex remains the same. Angular momentum has been lost from the inner vortex. The energy that is lost shows up as heat in the outer vortex. Thus the outer vortex becomes warm and the inner vortex is cooled.

Counterflow vortex tubes can be purchased by Exair Corporation of Cincinnati, Ohio or any other reputable vortex tube manufacturer. Vortex tubes and their method of operation are well known, such tubes are described, for example, in expired Fulton U.S. Pat. Nos. 3,173,273 and 3,208,229, and Ranque U.S. Pat. No. 1,952,281 (which are hereby incorporated by reference in this application). Compressed air (or other gas) from any suitable source enters such a tube and is throttled through nozzles to produce the special temperature change effects which are the unique characteristics of a vortex tube. The result is that the compressed air entering the tube is divided into hot and cold fractions from outlets at opposite ends of the tube. Usually a vortex tube is used for the cold air produced with typical temperatures at the cold air outlet ranging from minus 40° F. to plus 30° F. The air fraction discharged from the hot end is commonly exhausted to atmosphere.

In some applications, such as where a vortex tube is used for cooling the wearer of a protective suit, or a suit (or helmet) worn in a sandblasting operation or in some other industrial operation, some control over the extent of cooling is required to meet the needs or preferences of the wearer. Quite commonly, such control is achieved by providing a valve at the tube's hot end which may be manually adjusted to regulate the proportion of air discharged from the respective ends. Since the temperature reduction of the air discharged from the cold end of the vortex tube varies indirectly with the amount of air flowing therefrom, an adjustment which causes a greater proportion of the compressed air to escape from the cold end (and a lesser proportion from the hot end) would also result in an elevation of the temperature of air from the cold end. However, the reduction in the cooling effect resulting from an increase in the air temperature discharged from the tube's cold end may be offset at least partly by the increased volume of air flowing from the cold end. A user desiring to reduce the cooling effect and adjusting the vortex tube in order to increase the temperature of the air discharged from the tube's cold end might, because of such increased flow, sense that even further adjustment is necessary. Because manual adjustments produce changes in flow as well as temperature, a user may encounter considerable difficulty in selecting a condition of adjustment which provides just the right amount of cooling.

In expired U.S. Pat. No. 4,240,261 (which is hereby incorporated by reference in this application); Inglis discloses an alternative vortex tube—a counterflow vortex tube assembly. This assembly is equipped with a control which may be shifted into any selected position along its range of movement to produce corresponding or “sensible” changes in the temperature of the air discharged from the primary outlet of the assembly; having an operating handle which may be shifted in any selected position of adjustment to control the temperature of the air discharged from the primary outlet without at the same time substantially altering the rate of flow from that outlet; equipped with a single operating handle which may be readily adjusted to vary the temperature of the air discharged from the primary outlet over a temperature range extending from maximum hot to maximum cold; provide a “sensible” control for regulating the temperature of air discharged from a vortex tube; that is, a control which may be shifted into settings anywhere from one extreme position to the other to produce corresponding changes in discharge temperature bearing a generally linear relation with respect to the settings of the control handle (a.k.a. valve 6).

The assembly can comprise a basic vortex tube mounted within a housing having a primary outlet and a secondary outlet. A pair of flow-dividing members is disposed within the chamber of the housing, one of the members being movable with respect to the other to regulate the proportions of cold and hot air passing into the respective outlets.

More specifically, a first flow-dividing member is disposed within the chamber of the housing and is provided with a hot air passage receiving air from the hot end of the vortex tube and a cold air passage receiving air from the tube's cold end. A second flow-dividing member is disposed immediately adjacent the first and is provided with a pair of flow passages extending therethrough. One of such flow passages of the second flow-dividing member is in continuous communication with the primary outlet of the housing whereas the other flow passage remains in communication with the secondary outlet. The members are positioned and arranged so that the two flow passages of the second member receive substantially all of the air passing from the hot and cold air passages of the first member, the relative positions of the two members determining the proportions of hot and cold air which each flow passage of the second member receives from the passages of the first member.

The two members are generally cylindrical and are coaxial with respect to each other and to the vortex tube itself. The first flow-dividing member is provided with an axial recess which rotatably receives an end portion of one outlet tube, preferably the hot air tube, of the vortex tube. The second flow-dividing member is fixed within the housing with opposing end surfaces of the two members in slidable sealing engagement with each other. By rotating the first member relative to the second member so that the flow passage of the latter which communicates with the primary outlet of the assembly receives varying amounts of air from the hot and cold passages of the first member, the temperature of the air discharged from the primary outlet may be varied without significantly altering the volume of air flowing through that outlet.

In U.S. Pat. No. 3,173,275 (which is hereby incorporated by reference in this application), Fulton discloses that the cold end of a vortex tube can be directly applied to any part of a human patient. The body part must be directly applied to the aperture of the cold end and form a requisite seal with the aperture. See col. 13, lines 35-38. The resulting effect of the body part is that it forms a white spot and is at least −50° F.

b. Medical Applications

It is well known in the medical art that depriving the brain of blood for even a short period of time results in irreversible damage to the brain tissue. Such deprivation occurs during stroke, respiratory arrest, cardiac arrest, trauma and other severe bodily disturbances that slow or otherwise hinder the flow of oxygenated blood to the brain. However, it is also known that lowering the temperature of the brain (hypothermia) slows its metabolic activity, and reduces the chance of tissue damage when the oxygenated blood supply is diminished.

At present, operative neurosurgery and cardiac surgery is done in many cases using hypothermia for the specific purposes of maintaining cerebral and cardiac function. In an operating room, this requires use of a cooling module in conjunction with heart/lung bypass techniques by which the patient's blood, and resultantly the patient's brain tissue, is cooled. This widespread ability to rapidly lower brain temperature by as little as four or five degrees can make an enormous difference in preservation of neurological function and outcome. However, out in the field, when medical emergencies occur, brain cooling must quickly and expeditiously take place without access to the sophisticated equipment available in the hospital operating room. A portable brain cooling apparatus usable in the field is described in U.S. Pat. No. 5,261,399, issued in the names of Klatz et al. For use on an injured or disabled patient, Klatz et al. disclose a helmet and back plate containing cavities in which a coolant flows to cool the brain by means of heat conduction through the skull and upper spinal column. Other similar apparatuses are disclosed in U.S. Pat. Nos. 5,916,242; 4,566,455; 4,750,493; 4,763,866; 4,020,963; 5,190,032; 5,486,204; 5,643,336; 5,897,581; 5,913,855; 5,057,964; 6,030,412.

Schwartz wrote; in U.S. Pat. No. 5,916,247; “Rather than cooling the brain by the relatively slow heat conduction through the low heat conductivity of the bony skull and hair covering the head, the present invention teaches the use of a light weight, easily applied neck encircling collar in firm contact with the soft tissue of the neck, and particularly in good thermal contact with the carotid arteries traversing the neck. A coolant flowing through channels embedded in the collar rapidly cools the blood flowing through the carotid arteries which branch into blood vessels throughout the brain providing vascular access and attendant rapid internal cooling throughout the brain including its deepest recesses. Placing the collar on the patient's neck is easily and quickly accomplished simultaneously with other emergency medical techniques, such as CPR, which maintain the patient's heart and lung activity. [Schwartz'] collar contains no metallic parts; the collar, including the coolant channel, may be non-metallized fabric or plastic. This allows X-ray, CT scan, or MRI procedures to be used while the collar is in place without impairing the effectiveness of the procedure. In a second embodiment for rapid internal cooling of the brain, [Schwartz proposes] a conventional endotracheal tube, inserted into the trachea, is provided with a toroidal bladder surrounding the tube. The toroidal bladder is positioned at the back of the oral cavity, and a coolant flowing through the toroid cools blood vessels in the oral cavity which also traverse the brain, providing cooling of the brain tissue. The coolant flowing through the channels of the collar or the toroidal bladder may be any of the well known liquid or gaseous refrigerants, for example, gaseous CO₂, freon, or ice water, pumped through the channels of the collar or toroidal bladder in a manner known in the refrigeration art.”

Gaymar Industries, Inc., the assignee of this application, is the manufacturer of T-pad™ bladders. These bladders can receive a fluid, circulate the fluid within the interior of the bladder, and release the fluid to a fluid source or fluid receiver through an outlet conduit, and/or ambient air if there are apertures spaced throughout the bladder and preferably directed toward the user of the bladder. The fluid can be a liquid and/or gas. The fluid is preferably of a desired temperature. The temperature can be altered, for example, by a Medi-Therm II fluid temperature device, or equivalents thereof. These bladders have been positioned en over a patient's carotid artery, carotid arteries, forehead, neck, hand(s), groin, leg(s), foot (feet), and various other parts of a patient's body. That way, the patient's body core temperature and temperature of various parts of the body can be increased, maintained and/or decreased.

Variations of Gaymar's T-pad unit are disclosed by Golden in U.S. Pat. No. 4,846,176. That patent discloses inserting metal materials, like metallic conductive plates and/or rivets, into the bladders. The metal materials conductively transmit the thermal energy from the fluid circulating through the bladder at a more efficient rate to the user. The metal materials should contact the user and the fluid.

In some medical situations, it may be desirable to heat the patient. An example of at least one medical situation is directed to heating cancerous cells. Accordingly, it may be desired to be able to heat portions of the body in an emergent situation.

These various cooling devices require cold fluids that may not be present or inconvenient to maintain in an ambulance or a hospital setting. The present invention solves this problem by utilizing products that are already present in the hospital and/or ambulance settings—like piped compressed fluids or bottled compressed fluids—, and simultaneously not intentionally damage the patient's skin by freezing or burning as suggested in the prior art.

### SUMMARY OF THE INVENTION

The present invention is directed to a vortex tube assembly. The vortex tube assembly has a compressed fluid inlet. When the compressed fluid circulates in the vortex tube, the compressed fluid forms a cold end on the vortex tube that expels cold thermal energy and a warm end on the vortex tube that expels warm thermal energy. The present invention also has an applicator attached to a vortex tube to distribute thermal energy from the vortex tube to a patient to increase or decrease at least a portion of the patient's temperature without damaging the patient's skin.

### BRIEF DESCRIPTION OF THE DRAWINGS

FIG. 1 illustrates an embodiment of the prior art.

FIG. 2 illustrates the present invention taken from line 2-2 of FIG. 1.

FIG. 3 illustrates the present invention taken from line 3-3 of FIG. 1.

FIG. 4 illustrates a variation of FIG. 2.

FIG. 5 illustrates a variation of FIG. 3.

FIG. 6 illustrates a variation of FIG. 2.

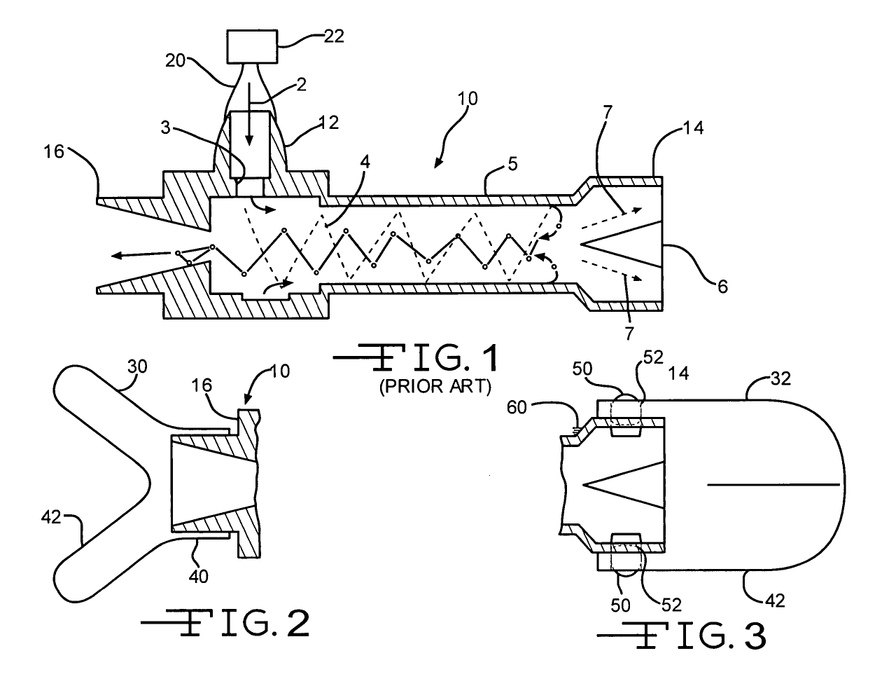

The embodiments illustrated in FIGS. 2-6 are interchangeable with either end of the vortex tube or a vortex tube having a switching mechanism to vary fluid flow temperature.

### DETAILED DESCRIPTION OF THE PRESENT INVENTION

The present invention is directed to a new design and/or an add-on component for a vortex tube 10 and a method to use the same. By no means is Applicant altering the mechanics of a vortex tube that has been used in the prior art for numerous years. Rather Applicant is utilizing the characteristics of the vortex tube for medical purposes and/or in emergent situations by adapting the vortex tube for such purposes.

The operation of a vortex tube is described above and is relied herein. For this application, we need to know the vortex tube 10 has a compressed gas inlet 12, a warm end 14 and a cold end 16. The vortex tube 10 receives compressed gas through the inlet 12. The compressed gas is delivered to the inlet 12 through a conduit 20 from a compressed gas source 22. The compressed gas source 22 can be any conventional source—a tank, a compressor, an outlet from a remote tank or combinations thereof. Hospitals and ambulances normally have compressed gas sources. For most applications, the compressed gas can be any fluid that obtains the desired features of the vortex tube. That compressed material is a gas and is normally air because it is the cheapest compressed gas. The pressure of the air supplied to the vortex tube through compressed gas inlet 12 can be any pressure that will obtain the desired results. It has been found by some manufacturers of vortex tube that the pressure of the fluid can fall within the range of about 30 to 120 psig.

As previously stated, the compressed gas enters the vortex tube 10 and generates a cold stream 16 and a warm stream 14 of the vortex tube 10. For purposes of fully disclosing the present invention, it is believed sufficient to state that the vortex tube 10 operates to divide a stream of compressed air (or other gas(es)) entering the body of the tube through inlet 12 into hot and cold fractions, the hot fraction being discharged axially from the free end of outlet tube 14 and the cold fraction being discharged from the free end of outlet tube 16. By controlling the relative dimensions of the parts, the proportions of the respective fractions, and the maximum/minimum temperatures of those fractions, may be established as desired. Preferably, vortex tube 10 should be constructed so that the rate of discharge from the hot and cold ends is approximately equal.

If desired, a suitable noise muffling element, such as fine-mesh folded screening, may be mounted within and/or on the vortex tube 10. The screening not only provides a noise muffling function but also distributes the hot air more evenly through the vortex tube 10. Such a muffling element is in the public domain.

The vortex tube 10 of the present invention has a cold applicator 30 attached to the cold end 16 of the vortex tube 10 and/or a heat applicator 32 attached to the warm end 14 of the vortex tube as illustrated in FIGS. 2-6. The applicators 30, 32 are female components that attach to the respective ends of the vortex tube 10. The applicators 30, 32 form the requisite seal with the ends 14, 16.

The cold applicator has two parts. The first part is the interconnector 40. The interconnector 40 is designed to at least fit over the cold end 16 of the vortex tube 10. The second part is the thermal transfer unit 42. The thermal transfer unit 42 protrudes from the interconnector 40 and is shaped like the letter “U”, “V” or variation thereof. The thermal transfer unit 42 can be a solid material, a hollow tube, hollow tube with apertures 68 (FIG. 4) or a combination thereof. The thermal transfer unit 42 should preferably be a conductive material to effectively transfer the cold thermal energy from the cold end 16 of the vortex tube 10 to the patient.

The thermal transfer unit 42 is designed to be positioned over, in the preferred embodiment, both carotid arteries of a patient. Why the carotid arteries? Because the cooling the blood in the carotid arteries could effectively cool the remainder of the body core temperature and should effectively cool the temperature in the brain for the reasons set forth above. The thermal transfer unit 42 can also be positioned over other portions of a person's body to effectively cool the body core temperature. For example, the thermal transfer unit 42 can be placed over the wrists, soles of the feet, appendage joints or ankles of the person.

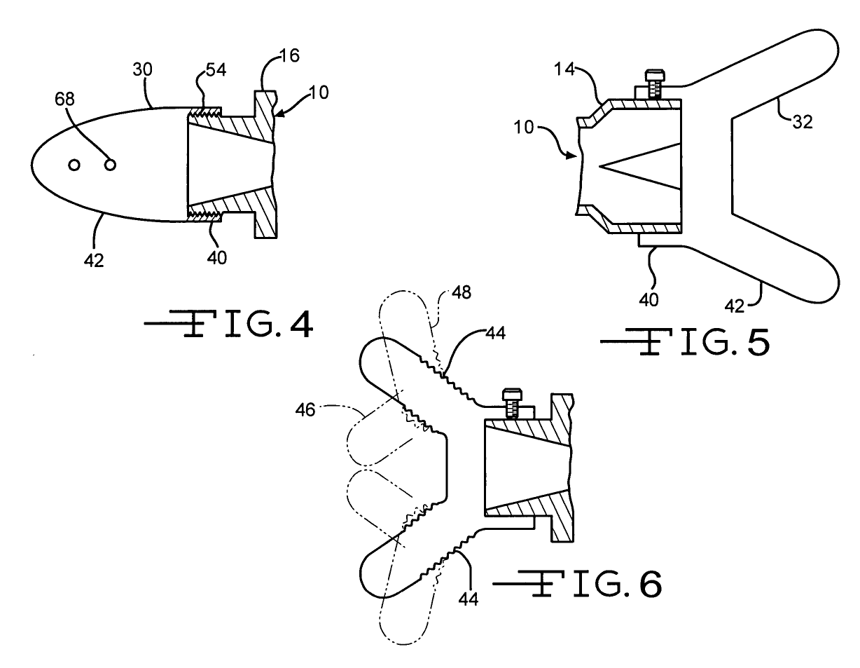

The thermal transfer unit 42 is conductive material or a material that provides for the diffusion of the hot and/or cold gas stream from the vortex tube for distribution over a predetermined portion of a patient's skin. The conductive material can be metallic material, polymeric conductive material, and combinations thereof. The composite material (a.k.a, combinations thereof) could be a product called MetaFor which is a porous metal/polymer hybrid material. The thermal transfer unit can be flexible to fit over various sized necks, ankles, appendage joints and wrists of a patient. The thermal transfer unit can have accordion-like features 44 that allows the thermal transfer unit to become narrower 46 or wider 48 for various sized applications as illustrated in FIG. 6. Alternatively, the thermal transfer unit 42 can be a specific shape and size. If the alternative embodiment is used, there can be various sized cold applicators 30 that interconnect to the cold end 16 of the vortex tube.

Alternatively, the thermal transfer unit 42 can be any shape that can apply cold thermal energy to any desired part of a patient's body. For example, it could be shaped to be straight, like the letter “I”, so it can be used in the patient's mouth to cool the tongue, or anyplace else to cool that body part or other parts of the body.

The heat applicator 32 is identical to the cold applicator 30, except that the heat applicator fits over the warm end 14 of the vortex tube 10 and allows the distribution (and sometimes referred to as the transfer) of the warm thermal energy from the warm end 14 of the vortex tube 10 to the patient. In some cases the heat applicator 32 can be interconnected to the cold applicator 30 so the thermal energies of the respective ends can converge and intermix with each other to obtain a desired intermediate temeperature.

The ends 14, 16 mate with the respective applicators 30, 32 by various conventional means. The components 14,16 and 30,32 can (1) slip together tightly or, alternatively, loosely (FIG. 2); (2) be snapped together through an aperture 52 on one component and an actuating locking mechanism 50 on the other respective component (FIG. 3); (3) be crimped together; (4) be threaded 54 together (FIG. 4); (5) twisted and snapped together, (6) twisted and hooked together; (7) welded together (conventional welding if metal or sonic or heat welded if other materials are used); (7) at least one screw 56 could bind the components together (FIG. 6); (8) be adhered together by an adhesive.

As previously suggested in the prior art, the vortex tube can be adjusted to provide the desired thermal energy. The present invention can have indicia 60 (FIG. 3) on the vortex to assist the user of the medically modified vortex tube select the desired thermal energy.

The medically altered vortex tube 10 is interconnected to a compressed gas that is found within an emergency situation. The emergency situation may be with an emergency medical technician, a firefighter, a medical provider (a doctor or a nurse), a policeman or any thing similar. The user of the device interconnects, unless the applicator is permanently attached to the vortex tube, the desired applicator(s) 30, 32 to the desired end(s) 14, 16 of the vortex tube 10; interconnects the compressed gas to the vortex tube; and applies an applicator to a desired part of a patient's body to receive the desired thermal energy without damaging the skin with either thermal energy (warm or cold) and in particular not forming white spots on the skin when the cold thermal energy is applied to the skin.

The present invention has been directed to compressed gases. The vortex tubes can also use other fluids that pass through the vortex tubes that obtain the desired cooling and heating of conventional vortex tubes.

While in the foregoing we have disclosed an embodiment of the invention in considerable detail for purposes of illustration, it will be understood by those skilled in the art that many of these details may be varied without departing from the spirit and scope of the invention.

We claim:

1. A vortex tube assembly having a compressed fluid inlet, and when the compressed fluid circulates in the vortex tube the compressed fluid forms (a) a cold end on the vortex tube that expels cold thermal energy and (b) a warm end on the vortex tube that expels warm thermal energy; the vortex tube assembly comprising:
an applicator attached to the cold end of the vortex tube to distribute the cold thermal energy from the cold end to a patient to decrease at least a portion of the patient's temperature without forming white spots on the patient.

2. The vortex tube of claim 1 wherein the vortex tube is temperature-adjustable.

3. The vortex tube of claim 1 wherein the applicator is removably attached to the cold end of the vortex tube.

4. The vortex tube of claim 1 wherein the applicator is permanently attached to the cold end of the vortex tube.

5. The vortex tube of claim 1 wherein the applicator has an interconnector that attaches to the cold end and a thermal transfer unit that conducts the cold thermal energy to the patient and extends from the interconnector.

6. The vortex tube of claim 5 wherein the thermal transfer unit is shaped to contact one portion of a patient.

7. The vortex tube of claim 5 wherein the thermal transfer unit is shaped to contact at least two portions of a patient.

8. The vortex tube of claim 7 wherein the thermal transfer unit is flexible.

9. The vortex tube of claim 7 wherein the thermal transfer unit is inflexible.

10. The vortex tube of claim 1 wherein the applicator attaches to the vortex tube and is selected from the group consisting of slipped together; snapped together through an aperture on one component and an actuating locking mechanism on the other respective component; crimped together; threaded together; twisted and snapped together; twisted and hooked together; welded together; screwed together; and adhered together.

11. The vortex tube of claim 2 that has indicia thereon to determine the temperature of the cold thermal energy protruding from the cold end.

12. A vortex tube assembly having a compressed fluid inlet, and when the compressed fluid circulates in the vortex tube the compressed fluid forms (a) a cold end on the vortex tube that expels cold thermal energy and (b) a warm end on the vortex tube that expels warm thermal energy; the vortex tube assembly comprising:
an applicator attached to the warm end of the vortex tube to distribute the warm thermal energy from the warm end to a patient to increase at least a portion of the patient's temperature without damaging the patient's skin.

13. The vortex tube of claim 12 wherein the vortex tube is temperature-adjustable.

14. The vortex tube of claim 12 wherein the applicator is removably attached to the warm end of the vortex tube.

15. The vortex tube of claim 12 wherein the applicator is permanently attached to the warm end of the vortex tube.

16. The vortex tube of claim 12 wherein the applicator has an interconnector that attaches to the warm end and a thermal transfer unit that conducts the warm thermal energy to the patient and extends from the interconnector.

17. The vortex tube of claim 16 wherein the thermal transfer unit is shaped to contact one portion of a patient.

18. The vortex tube of claim 16 wherein the thermal transfer unit is shaped to contact at least two portions of a patient.

19. The vortex tube of claim 16 wherein the thermal transfer unit is flexible.

20. The vortex tube of claim 16 wherein the thermal transfer unit is inflexible.

21. The vortex tube of claim 12 wherein the applicator attaches to the vortex tube and is selected from the group consisting of slipped together; snapped together through an aperture on one component and an actuating locking mechanism on the other respective component; crimped together; threaded together; twisted and snapped together; twisted and hooked together; welded together; screwed together; and adhered together.

22. The vortex tube of claim 13 that has indicia thereon to determine the temperature of the warm thermal energy protruding from the warm end.

23. The vortex tube of claim 13 wherein the compressed fluid is air.

24. The vortex tube of claim 1 wherein the compressed fluid is air.

23. A method of using a vortex tube assembly having a compressed fluid inlet; when the compressed fluid circulates in the vortex tube the compressed fluid forms (a) a cold end on the vortex tube that expels cold thermal energy and (b) a warm end on the vortex tube that expels warm thermal energy; an applicator attached to the warm end or the cold end and designed to distribute the thermal energy of the respective end to a patient; comprising
interconnecting a compressed fluid to the vortex tube through the fluid inlet; and
applying the applicator to a desired part of a patient's body to increase or decrease at least a portion of the temperature of the patient without damaging the patient's skin.

24. The method of claim 23 further comprising the step of attaching the applicator to the vortex tube.

25. The method of claim 24 wherein the applicator is permanently attached to the vortex tube.

26. The method of claim 24 wherein the applicator is removably attached to the vortex tube.

27. The method of claim 23 wherein the applicator contacts at least two portions of the patient's skin.

28. The method of claim 27 wherein the applicator is adjusted to contact the at least two portions of the patient's skin.

29. The method of claim 23 wherein the compressed fluid is air.

30. The method of claim 23 wherein the vortex tube can be adjusted to provide different thermal energies from the cold end and the warm end.

31. The method of claim 30 wherein the adjustments are made in accordance with temperature indicia on the vortex tube.
